(19) United States
(12) Patent Application Publication
(10) Pub. No.: US 2004/0073099 A1
(43) Pub. Date: Apr. 15, 2004

(54) Brain fluid ion concentration modification for treating neurological disorders

(75) Inventors: Johan F.M. Gijsbers, Geleen (NL); Frans L.H. Gielen, Eckelrade (NL)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN

(21) Appl. No.: 10/644,106

(22) Filed: Aug. 20, 2003

Related U.S. Application Data
(63) Continuation-in-part of application No. 10/174,257, filed on Jun. 18, 2002.
Application No. 10/174,257 is a continuation of application No. 09/561,550, filed on Apr. 28, 2000, now Pat. No. 6,447,500.
(60) Provisional application No. 60/404,605, filed on Aug. 20, 2002.

(51) Int. Cl. A61B 5/00

(52) U.S. Cl. 600/309

(57) ABSTRACT

Medical disorders with a neurological origin may be treated by modifying the ion concentration of brain fluid in the brain of a patient. The ion concentration of the fluid of the brain may be modified by delivering fluid to the brain, the ion concentration of the delivered fluid being such as to cause the ion concentration in the brain to be modified. The method may include replacing brain fluid, and may include replacing such fluid with fluid previously extracted from the brain. Epilepsy and other neurological disorders that are affected by the electrical potential difference between intracellular fluid and extra-cellular fluid and therefore the cell membrane potentials, and therefore the thresholds for the communication between brain cells can be controlled by re-circulating extra cellular brain fluid after the fluid has been treated to alter its ion concentrations. A computer-controlled pump can precisely control the extraction and delivery of brain fluid after the ion concentration of the fluid is appropriately adjusted, e.g. guided by the Goldman equation. Well-known techniques for modifying ion concentrations can be used to raise or lower ion concentrations as needed.

### RELATED APPLICATIONS

This patent application is a continuation-in-part of continuation of U.S. patent application Ser. No. 10/174,257, filed Jun. 18, 2002, which is a continuation of U.S. Ser. No. 09/561,550, filed Apr. 28, 2000, now U.S. Pat. No. 6,447,500 B1, issued Sep. 10, 2002, for which priority is claimed and these priority applications are incorporated herein by reference in their entireties. This patent application also claims priority to U.S. Provisional Patent Application Serial No. 60/404,605, filed Aug. 20, 2002, which is also incorporated herein by reference in its entirety.

### FIELD OF THE INVENTION

This invention relates to methods of treating medical disorders. In particular, this invention, which is rooted in the basic concepts described by the Goldman equation, relates to a method of treating the cause of epilepsy. This equation describes the relation between the cell rest membrane potential and the concentration of ions inside and outside the cells in e.g. nervous and muscle tissue. This implies that cell excitability can be modified and therefore the physiological inter-connectivity between cells. This interconnectivity is a major factor in the generation of e.g. epilepsy. The key concept of this invention is that if cell rest membrane electrical potentials are modified, epilepsy, and perhaps other neurological disorders might be effectively controlled.

### BACKGROUND OF THE INVENTION

Epilepsy is a debilitating neurological disorder. Functional control of many or all body functions can be lost and further, in general permanent brain damage results from each generalized epileptic attack.

It is known that an epileptic seizure is manifested by an uncontrolled propagation of nerve impulses throughout the nerve cells in certain, areas of the brain. The nerve impulses of an epileptic seizure are characterized by many synchronized discharges, which may involve the whole brain. As a consequence the control of many body function is lost. During epileptic seizures, the normal physiological interconnectivity between brain cells is greatly altered, resulting in a synchronized highly pathological brain activity.

It is well known that the normal, electrical, rest membrane potential difference between intra-cellular fluid (fluid enclosed by the cell membrane) of brain cells and the extra-cellular brain fluid (fluid outside the membrane) is about −0.07 volts (−70 millivolts or mV.) The intra-cellular brain fluid is at a more negative potential than the extra cellular fluid potential. If this potential becomes more negative (cells are hyper polarized), the likelihood of an epileptic seizure is decreased. In the field of biophysics, the well known Goldman equation describes how the membrane potential depends on the concentrations of ions in the intra- or extra-cellular medium. Consequently this equation describes how changes in the extra-cellular ion concentrations will result in a hyper-polarization of brain cells which will result in suppression of epileptic seizures.

### SUMMARY OF THE INVENTION

A method of treating medical disorders comprises the step of modifying the ion concentration of brain fluid in the brain of a patient. The method may be focused on neurological disorders, and may also comprise multiple additional steps.

The ion concentration of the fluid of the brain may be modified by delivering fluid to the brain, the ion concentration of the delivered fluid being such as to cause the ion concentration in the brain to be modified. The method may include replacing brain fluid, and may include replacing such fluid with fluid previously extracted from the brain. The step of delivering fluid to the brain may include delivering modulated ion-content or ion-adjusted fluid into the patient's brain into at least one localized region of the patient's brain, as for example, by pumping modulated ion-content fluid into the patient's brain according to a predetermined schedule of flow rates. Alternatively, the step of delivering fluid to the brain includes delivering modulated ion-content fluid into the patient's brain into a general region of the patient's brain.

The brain fluid's ion concentration may be measured. The measuring of ion concentration in said brain fluid may begin after the beginning of delivery of fluid to the brain, and the ion concentration of the delivered fluid may be adjusted based on the measured ion concentration. The method may be practiced using closed-loop feedback. Any pump may be computer-controlled.

Ion concentration in the brain fluid may be calculated using a membrane potential equation. For example, the membrane potential in the brain fluid may be calculated using the Goldman equation or using a derivative or modification of the Goldman equation. The delivery of modulated ion-content fluid may be adjusted, based on the desired membrane potential change. The electrical conductivity of said brain fluid may be measured after delivering modulated ion-content fluid to the patient's brain. The delivery of modulated ion-content fluid may be adjusted, based on the measured electrical conductivity of said brain fluid.

Preferably, the delivery is of modulated ion-content fluid and the delivered fluid produces a voltage differential, predicted by the Goldman equation, between intra-cellular fluid and extra-cellular fluid, which may be modified to such a level that epileptic seizures are controlled. The most likely epileptic brain cells may be predetermined, and the method may comprise the step of adjusting the delivery of modulated ion-content fluid based upon the measured electrical activity of these most likely epileptic brain cells. An epileptic condition in a patient may be diagnosed using any suitable apparatus and/or method, which are well known the art.

Fluid may be delivered to the brain using a dispersed delivery system, delivered to the brain ventricle, and delivered at a predetermined location by direct injection into a localized region.

A method of treating epilepsy whereby seizures can be suppressed or prevented by using extra-cellular fluid (in the central nervous system, cerebral spinal fluid or “CSF”) that is extracted from the brain, e.g. from one of the brain ventricles. The extracted brain fluid is treated to change the concentration of ions in the fluid in such a way that cells surrounded by this modified fluid will be hyper-or hypo-polarized which is quantitatively predicted by the Goldman equation. The ion-adjusted fluid is re-injected into the brain into a specific brain structure, which may contain the brain cells that generate the epileptic seizure (e.g.: hyper-polarization needed) or in a brain structure that modulates the epileptic region (hyper-polarization needed for suppressing structures and hypo-polarization needed for activating structures). The increased negative potential difference (hyper-polarization) between the intra and extra-cellular fluid in the epilepsy generating brain structure increases the potential difference that the nerve cells must overcome to be involved in the generation of an epileptic seizure. In effect, the invention includes modulating the interconnectivity of nerve cells by modulating the rest membrane electrical potential.

### BRIEF DESCRIPTION OF THE DRAWINGS

FIG. 1 shows a simplified block diagram of an apparatus that could perform the method disclosed and claimed herein.

FIG. 2 shows the steps of the method contemplated herein.

### DETAILED DESCRIPTION OF THE PREFERRED EMBODIMENTS

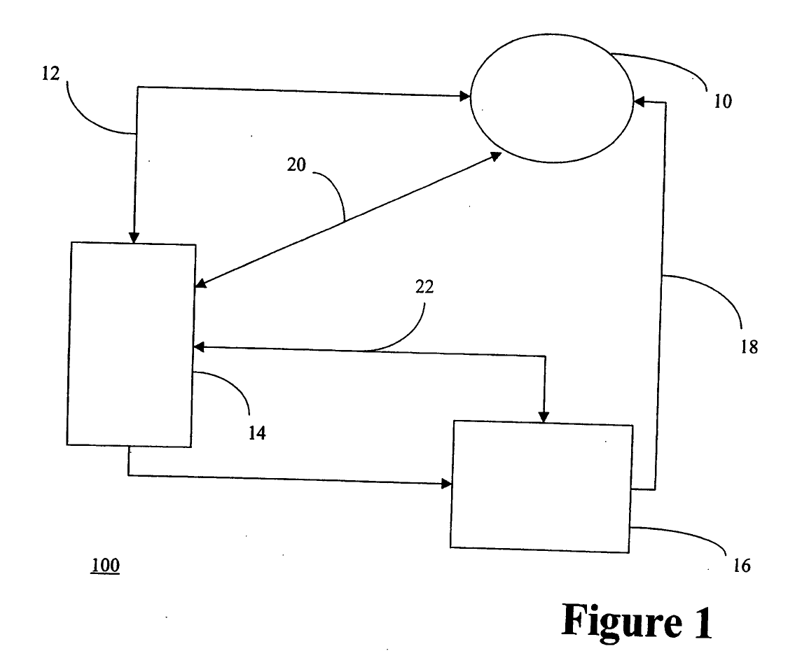

FIG. 1 shows a simplified schematic block diagram for a mechanical system for treating epilepsy and other neurological disorders by modifying ion concentration in brain fluid. In FIG. 1, fluid from a patient's brain 10 (the fluid is not shown) is extracted from the brain (preferably from 1 of the brain ventricles) 10, by a pump 14. Extra-cellular fluid can be extracted by exerting a relatively negative pressure on a small diameter flexible conduit (i.e. a catheter or capillary tube 12) one end of which is coupled to the pump and which provides the negative pressure. The other end of the flexible conduit is inserted into the brain ventricle. Extra-cellular brain fluid is drawn through an appropriately-sized capillary tube or catheter 12 to the pump 14, which in the preferred embodiment was a positive displacement computer-controlled pump 14.

In the preferred embodiment, the pump 14 (which may be computer controlled) reads and executes stored program instructions that cause the pump to pump the extracted fluid according to the program and its parameters. In many applications, ion-adjusted fluid will be pumped in an “open loop” fashion, i.e. according to some predetermined schedule in the pump's stored program. Open-loop delivery methods can be based upon either the volume of modified extra-cellular brain fluid to be delivered per unit time or some other parameter.

The pump 14 forces extracted extra-cellular brain fluid through an ion concentration adjustment mechanism 16. Ion concentrations in the extracted brain fluid are modified in the ion concentration adjustment mechanism 16. The ion concentration of the fluid can be adjusted by methodologies well known in the art including, but not limited to, appropriate ion exchange mechanisms; filtration, or chemical treatment. The ion concentration adjustment mechanism 16 changes the ion concentration in brain fluid such that when the fluid is returned to the brain, the brain fluid ion concentration, at least in localized regions, is modified. Output from the ion concentration adjustment mechanism 16 is returned to the brain 10 through an appropriately sized capillary tube or catheter 18.

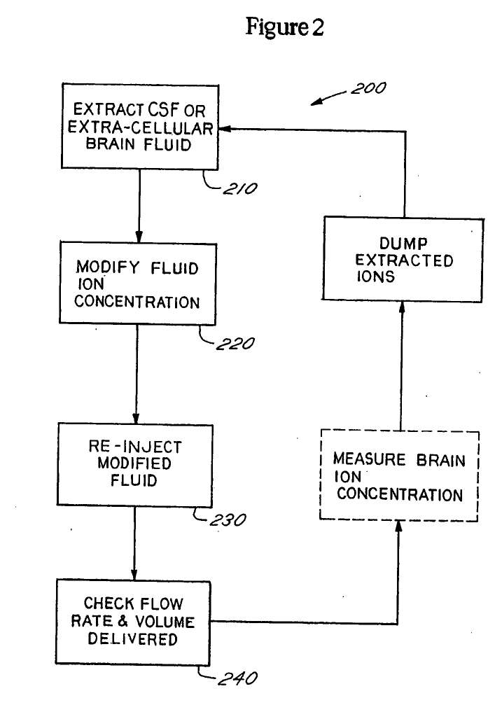

Procedural steps of the method 200 of the invention are illustrated in FIG. 2. CSF or extra-cellular brain fluid is extracted 210 and the ion-concentration of the fluid is adjusted 220 using an appropriate methodology. Some techniques for modifying ion concentration would include filtering or various chemical treatment processes. After the ion concentration is adjusted, the modified ion-content fluid is re-injected into the patient's brain 230.

At some point in the process, the flow rate of brain fluid from and/or into the patient's brain is measured 240. While this step is shown in FIG. 2 as being after re-injection of the brain fluid 230, alternate methodologies would certainly include deleting this step in its entirety and simply letting the pump run “open loop” doing whatever the program instructions dictate. Still other embodiments would include calculating or measuring the extracted fluid 210 volume as well as the delivered fluid volume. Inasmuch as brain fluid is incompressible, both the extracted and re-injected or delivered fluid volumes should be equivalent, except for any fluid lost during the ion-concentration adjustment.

Still other embodiments might measure the ion concentration in the brain fluid and, depending upon the measured ion concentration, adjust the fluid delivery rate or the ion concentration adjustment, or both. In an optimum system, a closed-loop feedback system would include a system that measures ion concentration (or other electrical characteristic) and uses this information to control fluid extraction, delivery or ion content so as to achieve the optimum electrical potential difference between the inside and outside of brain cells, imbalances of which might cause epilepsy or other neurological disorders.

Various embodiments of the invention include localized delivery of ion-odulated brain fluid as well as dispersed delivery mechanism, such as a leaky catheter. By replacing the brain fluid at a modified ion concentration, it is possible to change the electrical potential difference between intra-cellular and extra-cellular brain fluid.

In the preferred embodiment, changing the electrical potential difference across the nerve cell membrane in the epilepsy generating brain structure can significantly affect the occurrence of epileptic seizures. Once a diagnosis of epilepsy is made or the disease is established, changing the ion concentration in the extra-cellular fluid to increase the potential difference from −70 millivolts to −80 or more millivolts will locally hyper -polarize the brain cells and therefore, can substantially inhibit seizures.

In one of the alternate embodiments, an electrical probe 20 inserted into a localized region of the brain 10 might be read by the computer that controls the pump 14 so as to provide closed-loop feedback so as to even more closely control ion concentrations and therefore more closely control epileptic seizures. A probe inserted into the brain fluid in the brain might measure the ion concentration by the conductivity or resistance of the fluid. In such an embodiment, it is preferable to measure ion concentration after the ion adjusted fluid has been returned to the patient's brain. The modified resulting membrane potential can be calculated using the well-known Goldman equation. In another alternate embodiment, ion concentrations of extra-cellular fluid might be adjusted according to measured electrical activity of nerve cells in specific brain structures involved in the generation of the epileptic seizures. In such an embodiment, the electrical activity of brain cells, can be continuously adjusted by injecting more or less modified brain fluid in such a way to avoid seizures. Such a closed-loop system could be used to carefully control, in real time, the rate at which ion adjusted fluid is delivered to the brain or to change the ion concentration changes effected by the ion concentration adjustment mechanism 16. A control signal 22 from the computer-controlled pump 14 might be used to change the ion concentration in fluid that is output to the brain 10. In yet another embodiment, it might be possible to alter electrical potential differences across cell membranes simply by adding or administering a predetermined liquid or pharmacological agent or other substance to brain fluid so as to change the electrical potential across brain cells.

Those skilled in the art will recognize that changing the ion concentration of extra cellular brain fluid could have other beneficial effects in the treatment of other neurological disorders by adjusting the degree of communication between brain cells. This degree of communication depends on the level of the membrane potential. In general hyper polarized or inhibited cells increase the threshold for cell communication, while hypo polarized or excited cells decrease the threshold for cell communication. In instances where neurological disorders can be controlled by modulating the communication between brain cells the invention would find the applicability in treating these other disorders.

The following discussion is adapted from Eric R. Kandel et al., Principles of Neural Science (4^{th} Edition, 2000), Chapter 7. The present invention includes the use of the concepts and various equations described in Kandel et al., which is incorporated herein by reference.

### Membrane Potential

Information is carried within and between neurons by electrical and chemical signals. Transient electrical signals are particularly important for carrying time-sensitive information rapidly and over long distances. These electrical signals-receptor potentials, synaptic potentials, and action potentials—are all produced by temporary changes in the current flow into and out of the cell that drive the electrical potential across the cell membrane away from its resting value.

This current flow is controlled by ion channels in the cell membrane. We can distinguish two types of ion channels—resting and gated—by their distinctive roles in neuronal signaling. Resting channels normally are open and are not influenced significantly by extrinsic factors, such as the potential across the membrane. They are primarily important in maintaining the resting membrane potential, the electrical potential across the membrane in the absence of signaling. Most gated channels, in contrast, are closed when the membrane is at rest. Their probability of opening is regulated by the three factors: changes in membrane potential, ligand binding, or membrane stretch.

We consider how transient electrical signals are generated in the neuron. We begin by discussing how resting ion channels establish and maintain the resting potential. We also briefly describe the mechanism by which the resting potential can be perturbed, giving rise to transient electrical signals such as the action potential. Passive properties of neurons—their resistive and capacitive characteristics—contribute to local signaling within the neuron. Voltage-gated Na⁺, K⁺ and Ca²⁺ channels generate the action potential, the electrical signal conveyed along the axon. Synaptic and receptor potentials are a consideration in the context of synaptic signaling between neurons.

### The Resting Membrane Potential Results from the Separation of Charges Across the Cell Membrane

Every neuron has a separation of charges across its cell membrane consisting of a thin cloud of positive and negative ions spread over the inner and outer surfaces of the cell membrane. At rest a nerve cell has an excess of positive charges on the outside of the membrane and an excess of negative charges on the inside. This separation of charge is maintained because the lipid bilayer of the membrane blocks the diffusions of ions. The charge separation gives rise to a difference of electrical potential, or voltage, across the membrane called the membrane potential. The membrane potential (Vₘ) is defined as

Vₘ=Vᵢₙ=Vₒᵤₜ

where Vᵢₙ is the potential on the inside of the cell and Vₒᵤₜ the potential on the outside.

The membrane potential of a cell at rest is called the resting membrane potential. Since, by convention, the potential outside the cell is defined as zero, the resting potential (Vᵣ) is equal to Vᵢₙ. Its usual range in neurons is −60 mV to −70 mV. All electrical signaling involves brief changes from the resting membrane potential due to alterations in the flow of electrical current across the cell membrane resulting from the opening and closing of ion channels.

The electric current that flows into and out of the cell is carried by ions, both positively charged (cations) and negatively charged (anions). The direction of current flow is conventionally defined as the direction of net movement of positive charge. Thus, in an ionic solution cations move in the direction of the electric current, anions in the opposite direction. Whenever there is a net flow of cations or anions into or out of the cell, the charge separation across the resting membrane is disturbed, altering the polarization of the membrane. A reduction of charge separation, leading to a less negative membrane potential, is called depolarization. An increase in charge separation, leading to a more negative membrane potential, is called hyperpolarization. Changes in membrane potential that do not lead to the opening of gated ion channels, are called electronic potentials and are said to be passive responses of the membrane. Hyperpolarizing responses are almost passive, as are small depolarizations. However, when depolarization approaches a critical level, called the threshold, the cell responds actively with the opening of voltage-gated ion channels, which at threshold produces an all-or-none action potential.

The membrane potential can be examined by analyzing how the passive flux of individual ion species through resting channels generates the resting potential. This aids in the understanding of how the selective gating of different types of ion channels generates the action potential, as well as the receptor and synaptic potentials.

The Resting Membrane Potential is Determined by Resting Ion Channels

No single ion species is distributed equally on the two sides of a nerve cell membrane. Of the four most abundant ions found on either side of the cell membrane, Na⁺ and Cl⁻ are more concentrated outside the cell, and K⁺ and organic anions (A⁻) are more concentrated inside. The organic anions are primarily amino acids and proteins. Table I shows the distribution of these ions inside and outside one particularly well-studied nerve cell process, the giant axon of the squid, whose blood has a salt concentration similar to sea water. Although the absolute values of the ionic concentrations for vertebrate nerve cells are two-to threefold lower than those for the squid giant axion, the concentration gradients (the ratio of the external ion concentration to internal ion concentration) are about the same.

TABLE I
Distribution of the Major Ions Across a Neuronal
Membrane at Rest: The Giant Axon of the Squid

| | Concentration | Concentration | Equilibrium |
|---|---|---|---|
| Species | in cytoplasm | in extracellular | Potential¹ |
| of Ion | (mM) | fluid (mM) | (mV) |
| K⁺ | 400 | 20 | −75 |
| Na⁺ | 50 | 440 | +55 |
| Cl⁻ | 52 | 560 | −60 |
| A⁻(organic | 385 | — | — |
| anions) | | | |

¹The membrane potential at which there is no net flux of the ion species across the cell membrane.

The unequal distributional of ions raises several important questions, such as how do ionic gradients contribute to the resting membrane potential, and how are they maintained, and what prevents the ionic gradients from dissipating by diffusion of ions across the membrane through the passive (resting) channels. These questions are interrelated and can be answered by considering two examples of membrane permeability: the resting membrane of glial cells, which is permeable to only one species of ions, and the resting membrane of nerve cells, which is permeable to three. For the purposes of this discussion we shall consider only the resting ion channels, which are always open.

Resting Channels in Glial Cells are Selective for Potassium only

A membrane's overall selectivity for individual ion species is determined by the relative proportions of the various types of ion channels in the cell that are open. The simplest case is that of the glial cell, which has a resting potential of about −75mV. Here, the vast majority of the resting channels in the membrane are permeable only to K⁺. As a result, the glial cell membrane at rest is almost exclusively permeable to K⁺ ions. A glial cell has a high concentration of K⁺ and negatively charged organic anions on the inside and a high concentration of Na⁺ and Cl⁻ on the outside.

These ionic gradients generate the membrane potential of the glial cell. Because K⁺ ions are present at a high concentration inside the cell and glial cells are selectively permeable to them, K⁺ ions tend to diffuse from inside to outside the cell down their chemical concentration gradient. As a result, the outside of the membrane accumulates a positive charge (due to the slight excess of K⁺) and the inside a negative charge (because of the deficit of K⁺ and the resulting slight excess of anions). Since opposite charges attract each other, the excess positive charges on the outside and the excess negative charges on the inside collect locally on either surface of the membrane.

The diffusion of K⁺ out of the cell is self-limiting. The separation of charge resulting from the diffusion of K⁺ gives rise to an electrical potential difference:

positive outside, negative inside. The more K⁺ continues to flow, the more charge will be separated and the greater will be the potential difference. Since K⁺ is positively charged, this potential difference tends to oppose the further efflux of K⁺. Thus, ions are subject to two forces driving them across the membrane: (1) a chemical driving force that depends on the concentration gradient across the membrane and (2) an electrical driving force that depends on the electrical potential difference across the membrane. Once K⁺ diffusion has proceeded to a certain point, a potential develops across the membrane at which the electrical force driving K⁺ into the cell exactly balances the chemical force driving K⁺ ions out of the cell. That is, the outward movement of K⁺ (driven by its concentration gradient) is equal to the inward movement of K⁺ (driven by the electrical potential difference across the membrane). This potential is called the potassium equilibrium potential, E_{K}. In a cell permeable only to K⁺ ions, E_{K} determines the resting membrane potential, which in most glial cells is about −75 mV.

The equilibrium potential for any ion X can be calculated from an equation derived in 1888 from basic thermodynamic principles by the German physical chemist Walter Nernst:

$${E}_{x} = \frac{RT}{zF} ln \frac{{[ X ]}_{o}}{{[ X ]}_{i}} \qquad Nernst Equation$$

where R is the gas constant, T the temperature (in degress Kelvin), z the valence of the ion, F the Faraday constant, and (X)ₒ and (X)ᵢ are the concentrations of the ion outside and inside of the cell. (To be precise, chemical activities should be used rather than concentrations).

Since RT/F is 25 mV at 25° C. (room temperature), and the constant for converting from natural logarithms to base 10 logarithms is 2.3, the Nernst equation can also be written as:

$${E}_{x} = \frac{58 mV}{z} log \frac{[ {X}_{o} ]}{[ {X}_{i} ]}$$

Thus, for K⁺, since z=+1 and given the concentrations inside and outside the squid axon in Table I:

$${E}_{K} = \frac{58 mV}{1} log \frac{[ 20 ]}{[ 400 ]} = 75 mV$$

The Nernst equation can be used to find the equilibrium potential of any ion that is present on both sides of a membrane permeable to that ion (the potential is sometimes called Nernst potential). The Na⁺, K⁺ and Cl⁻ equilibrium potentials for the distribution of ions across the squid axon are given in Table I.

In our discussion so far we have treated the generation of the resting potential by the diffusion of ions down their chemical gradients as a passive mechanism, one that does not require the expenditure of energy by the cell, for example through hydrolysis of adenosine tri-phosphate (ATP). However, as we shall see below, energy (and ATP hydrolysis) is required to set up the initial concentration gradients and to maintain them during the activity of a neuron.

Resting Channels in Nerve Cells are Selective for Several Ion Species

Measurements of the resting membrane potential with intracellular electrodes and flux studies using radioactive tracers show that, unlike glial cells, nerve cells at rest are permeable to Na⁺ and Cl⁻ ions in addition to K⁺ ions. Of the abundant ion species in nerve cells only the large organic anions (A⁻)-negatively charged proteins and amino acids—are unable to permeate the cell membrane. The concentration gradients for the three permeant ions (Na⁺, K⁺, and Cl⁻) can be maintained across the membrane of a single cell, and these three gradients interact to determine the cell's resting membrane potential.

To understand this phenomena, it will be easiest to examine first only the diffusion of K⁺ and Na⁺, and return to the simple example of a cell having only K⁺ channels, with concentration gradients for K⁺, Na⁺, Cl⁻, and A⁻, as shown in Table I. Under these conditions the resting membrane potential, Vᵣ, is determined solely by the K⁺ concentration gradient and will be equal to E_{K} (−75 mV).

Now consider what happens if a few resting Na⁺ channels are added to the membrane, making it slightly permeable to Na⁺. Two forces act on Na⁺ to drive it into the cell. First, Na⁺ is more concentrated outside than inside and therefore it tends to flow into the cell down its chemical concentration gradient. Second, Na⁺ is driven into the cell by the negative electrical potential difference across the membrane. The influx of positive charge (Na⁺) depolarizes the cell, but only slightly from the K⁺ equilibrium potential (−75 mV). The new membrane potential does not come close to the Na⁺ equilibrium potential of +55 mV because there are many more resting K⁺ channels than Na⁺ channels in the membrane.

As soon as the membrane potential begins to depolarize from the value of the K⁺ equilibrium potential, K⁺ flux is no longer in equilibrium across the membrane. The reduction in the negative electrical force driving K⁺ into the cell means that there will be a net efflux of K⁺ out of the cell, tending to counteract the Na⁺ influx. The more the membrane potential is depolarized and moves away from the K⁺ equilibrium potential, the greater is the electrochemical force driving K⁺ out of the cell and consequently the greater is the K⁺ efflux. Eventually, the membrane potential reaches a new resting potential at which the outward movement of K⁺ just balances the inward movement of Na⁺. This balance point (usually −60 mV) is far from the Na⁺ equilibrium potential (+55 mV) and is only slightly more positive than the equilibrium potential for K⁺ (−75 mV).

To understand how this balance point is determined, bear in mind that the magnitude of the flux of an ion across a cell membrane is the product of its electrochemical driving force (the sum of the electrical driving force and the chemical driving force due to the concentration gradient) and the conductance of the membrane to the ion:

Ion flux=(electrical driving force+chemical driving force)×membrane conductance.

A cell has relatively few resting Na⁺ channels so at rest the conductance to Na⁺ is quite low. Thus, despite the large chemical and electrical forces driving Na⁺ into the cell, the influx of Na⁺ is small. In contrast, since there are many resting K⁺ channels, the membrane conductance of K⁺ is relatively large. As a result, the small net outward force acting on K⁺ at the resting membrane potential is enough to produce a K⁺ efflux equal to the Na⁺ influx.

Passive Flux of Sodium and Potassium is Balanced by Active Pumping of the Ions

For a cell to have a steady resting membrane potential the charge separation across the membrane must be maintained constant over time. That is, the influx of positive charge must be balanced by the efflux of positive charge. If these fluxes were not equal, the charge separation across the membrane, and thus the membrane potential, would vary continually. As we have seen, the passive movement of K⁺ out of the cell through resting channels balances the passive movement of Na⁺ into the cell. However, these steady ion leaks cannot be allowed to continue unopposed for any appreciable length of time because the Na⁺ and K⁺ gradients would eventually run down, reducing the resting membrane potential.

Dissipation of ionic gradients is prevented by the Na⁺—K⁺ pump, which moves Na⁺ and K⁺ against their net electrochemical gradients: it extrudes Na⁺ from the cell while taking in K⁺. The pump therefore requires energy to run. The energy comes from the hydrolysis of ATP. Thus, at the resting membrane potential the cell is not in equilibrium but rather in a steady state: there is a continuous passive influx of Na⁺ and efflux of K⁺ through resting channels that is exactly counterbalanced by the Na⁺—K⁺ pump.

The Na⁺—K⁺ pump is a large membrane-spanning protein with catalytic binding sites for Na⁺, K⁺, and ATP. The sites for Na⁺ and ATP are located on its intracellular surface and the sites for K⁺ on its extracellular surface. With each cycle the pump hydrolyzes one molecule of ATP. It then uses the energy to extrude three Na⁺ ions and bring in two K⁺ ions. The unequal flux of Na⁺ and K⁺ ions causes the pump to generate a net outward ionic current. Thus, the pump is said to be electrogenic. This pump-driven outward flux of positive charge tends to hyperpolarize the membrane to a somewhat more negative potential than would be achieved by the simple passive-diffusion mechanisms discussed above.

Chloride Ions May be Passively Distributed

So far in this discussion the contribution of chloride (Cl⁻ to the resting potential has been ignored, even though many nerve cells have Cl⁻ channels that are open in the resting membrane. This simplification is valid for nerve cells that do not have a mechanism for active transport of C⁻ against an electrochemical gradient. In these cells the resting potential is ultimately determined by K⁺ and Na⁺ fluxes because the intracellular concentrations of K⁺ and Na⁺ are fixed by active transport (the Na⁺—K⁺ pump), whereas the C⁻ concentration inside the cell is affected only by passive forces (electrical potential and concentration gradient). Therefore, the movement of C⁻ ions tends toward equilibrium across the membrane, so that E_{Cl} is equal to the resting potential, Vᵣ₉ and there is no net Cl⁻flux at rest.

In many nerve cells the Cl⁻ gradient is controlled by an integral membrane protein called a Cl⁻ transporter. Like the Na⁺, —K⁺ pump it catalyzes the movement of ions across the membrane against an electrochemical gradient without forming a continuous pore. Unlike the Na⁺—K⁺ pump, the transport process does not require the hydrolysis of ATP. Although no chemical bond energy is utilized in the transport process, the C⁻ transporter can move Cl⁻ against its electrochemical gradient by utilizing the energy stored in a preexisting ionic concentration gradient for a different type of ion—a process known as secondary active transport. For example, one type of Cl⁻ transporter couples the outward movement of one C⁻ ion to the outward movement of one K⁺ ion. Since the electrochemical gradient for K⁺ is outward, the energetically favorable outward K⁺ flux is able to drive the energetically unfavorable outward C⁻ flux. As a result, the outside-to-inside ratio of C⁻ is greater than would result from passive diffusion alone. The effect of increasing the C⁻ gradient is to make the equilibrium potential for C⁻ ions more negative than the resting membrane potential overall. (Remember, the valence (z) of C⁻ is −1.).

The Balance of Ion Fluxes that Gives Rise to the Resting Membrane Potential Is Abolished During the Action Potential

In the nerve cell at rest the steady Na⁺ influx is balanced by a steady K⁺ efflux, so that the membrane potential is constant. This balance changes, however, when the membrane is depolarized past the threshold for generating an action potential. Once the membrane potential reaches this threshold, voltage-gated Na⁺ channels open rapidly. The resultant increase in membrane permeability to Na⁺ causes the Na⁺ influx to exceed the K⁺ efflux, creating a net influx of positive charge that causes further depolarization. The increase in depolarization causes still more voltage-gated Na⁺ channels to open, resulting in a greater influx of Na⁺, which accelerates the depolarization even further.

This regenerative, positive feedback cycle develops explosively, driving the membrane potential toward the Na⁺ equilibrium potential of +55 mV:

$${E}_{Na} = \frac{RT}{F} ln \frac{{[ Na ]}_{o}}{{[ Na ]}_{l}} = 58 mV log \frac{[ 440 ]}{[ 50 ]} = + 55 mV .$$

However, the membrane potential never quite reaches that point because K⁺ efflux continues throughout the depolarization. A slight influx of C⁻ into the cell also counteracts the depolarizing tendency of the Na⁺ influx. Nevertheless, so many voltage-gated Na⁺ channels open during the rising phase of the action potential that the cell's permeability to Na⁺ is much grater than to either C⁻ or K⁺. Thus, at the peak of the action potential the membrane potential approaches the Na⁺ equilibrium potential, just as at rest (when permeability to K⁺ is predominant) the membrane potential tends to approach the K⁺ equilibrium potential.

The membrane potential would remain at this large positive value near the Na⁺ equilibrium potential indefinitely but for two processes that repolarize the membrane, thus terminating the action potential. First, as the depolarization continues, the population of voltage-gated Na⁺ channels gradually closes by the process of inactivation. Second, opening of the voltage-gated K⁺ channels causes the K⁺ efflux to gradually increase. The increase in K⁺ permeability is slower than the increase in Na⁺ permeability because of the slower rate of opening of the voltage-gated K⁺ channels. The delayed increase in K⁺ efflux combines with a decrease in Na⁺ influx to produce a net efflux of positive charge from the cell, which continues until the cell has repolarized to its resting membrane potential.

The Contributions of Different Ions to the Resting Membrane Potential can be Quantified by the Goldman Equation

Although Na⁺ and K⁺ fluxes set the value of the resting potential, Vₘ is not equal to either E_{K} or E_{Na} but lies between them. As a general rule, when Vₘ is determined by two or more species of ions, the influence of each species is determined not only by the concentrations of the ion inside and outside the cell but also by the ease with which the ion crosses the membrane. In terms of electrical current flow, the membrane's conductance (1/resistance) provides a convenient measure of how readily the ion crosses the membrane. Another convenient measure is the permeability (P) of the membrane to that ion in units of velocity, cm/s. This measure is similar to that of a diffusion constant, which measures the rate of solute movement in solution. The dependence of membrane potential on ionic permeability and concentration is given quantitatively by the Goldman equation.

$${V}_{m} = \frac{RT}{F} ln \frac{{{P}_{K} [ {k}^{+} ]}_{o} + {{P}_{Na} [ {Na}^{+} ]}_{o} + {{P}_{Cl} [ {Cl}^{-} ]}_{j}}{{P}_{K} [ {K}^{+} ] i + {{P}_{Na} [ {Na}^{+} ]}_{j} + {{P}_{Cl} [ Cl ]}_{o}}$$

This equation applies only when Vₘ is not changing. It states that the greater the concentration of a particular ion species and the greater its membrane permeability, the greater its role in determining the membrane potential. In the limit, when permeability to one ion is exceptionally high, the Goldman equation reduces to the Nernst equation for that ion. For example, if P_{K}>>P_{Cl} or P_{Na}, as in glial cells, the equation becomes

$$Vm = \frac{RT}{F} ln \frac{{[ {K}^{+} ]}_{o}}{{[ {K}^{+} ]}_{j}}$$

Alan Hodgkin and Bernard Katz used the Goldman equation to analyze changes in membrane potential. They first measured the variation in membrane potential of a squid giant axon while systematically changing the extracellular concentrations of Na⁺, Cl⁻, and K⁺. They found that if Vₘ is measured shortly after the extracellular concentration is changed (before the internal ionic concentrations are altered), [K⁺]ₒ has a strong effect on the resting potential, [Cl⁻]ₒ has a moderate effect, and [Na⁺]ₒ has little effect. The data for the membrane at rest could be fit accurately by the Goldman equation using the following permeability ratios:

P_{K}:P_{Na}:P_{Cl}=1.0:0.04:0.45.

At the peak of the action potential, however, the variation of Vₘ with external ionic concentrations was fit best if a quite different set of permeability ratios were assumed (at the peak of the action potential there is an instant in time when Vₘ is not changing and the Goldman equation is applicable):

P_{K}:P_{Na}:P_{Cl}=1.0:20:0.45.

For these values of permeabilities the Goldman equation approaches the Nernst equation for Na⁺:

$${V}_{m} = \frac{RT}{F} ln \frac{{[ {Na}^{+} ]}_{o}}{{[ {Na}^{+} ]}_{l}} = + 55 mV .$$

Thus at the peak of the action potential, when the membrane is much more permeable to Na⁺ than to any other ion, Vₘ approaches E_{Na}, the Nernst potential for Na⁺.

However, the finite permeability of the membrane to K⁺ and C⁻ results in K⁺ efflux and C⁻ influx that oppose Na⁺ influx, thereby preventing Vₘ from quite reaching E_{Na}.

The Functional Properties of the Neuron can be Represented in an Electrical Equivalent Circuit

The Goldman equation is limited because it cannot be used to determine how rapidly the membrane potential changes in response to a change in permeability. Moreover, it is inconvenient for determining the magnitude of the individual Na⁺, K⁺, and C⁻ currents. This information can be obtained with a simple mathematical model derived from electrical circuits. Within this model, called an equivalent circuit, all of the important functional properties of the neuron are represented by an electrical circuit consisting only of conductors or resistors (representing the ion channels), batteries (representing the concentration gradients of relevant ions), and capacitors (the ability of the membrane to store charge). Equivalent circuits provide us with an intuitive understanding as well as a quantitative description of how current flow due to the movement of ions generates signals in nerve cells. The first step in developing a circuit is to relate the membrane's discrete physical properties to its electrical properties.

Each Ion Channel Acts as a Conductor and Battery in Series

The lipid bilayer of the membrane is a poor conductor of ionic current because it is not permeable to ions. Even a large potential difference will produce practically no current flow across a pure lipid bilayer. Consider the cell body of a typical spinal motor neuron, which has a membrane area of about 10⁻⁴ cm². If the membrane were composed solely of lipid bilayer, its electrical conductance would be only about 1 pS. In reality, however, the membrane contains thousands of resting ion channels through which ions constantly diffuse, so that the actual conductance of the membrane at rest is about 40,000 pS or 40×10⁻⁹S, i.e., 40,000 times greater than it would be if no ion channels were present.

In an equivalent circuit each K⁺ channel can be represented as a resistor or conductor of ionic current with a single-channel conductance of γ_{K} (remember, conductance=1/resistance). If there were no K⁺ concentration gradient, the current through the K⁺ channel would be given by Ohm's law: i_{K}=γ_{K}×Vₘ. Since there is normally a K⁺ concentration gradient, there will be a chemical force driving K⁺ across the membrane. In a the equivalent circuit this chemical force is represented by a battery, whose electromotive force is given by the Nernst potential for K⁺, E_{K}. (A source of electrical potential is called an electromotive force and an electromotive force generated by a difference in chemical potentials is called a battery.)

In the absence of voltage across the membrane the normal K⁺ concentration gradient will cause an outward K⁺ current flow. According to our conventions for electrical current flow an outward movement of positive charge corresponds to a positive electric current. From the Nernst equation, we also saw that when the concentration gradient for a positively charged ion, such as K⁺, is directed outward (i.e., there is a higher K⁺ concentration inside than outside the cell), the equilibrium potential for that ion is negative. Thus, the K⁺ current that flows solely because of its concentration gradient is given by i_{K}=−γ_{K}×E_{K} (the negative sign is required because a negative equilibrium potential produces a positive current).

Finally, for a real neuron that has both a membrane voltage and K⁺ concentration gradient, the net K⁺ current is given by the sum of currents due to the electrical and chemical driving forces:

i_{K}=(γ_{K}×Vₘ)−(γ_{K}×E_{K})=γ_{K}×(Vₘ−E_{K})

The term Vₘ−E_{K} is called the electrochemical driving force. It determines the direction of ionic current flow and (along with the conductance) the magnitude of current flow. This equation is a modified form of Ohm's law that takes into account that ionic current flow through a membrane is determined not only by the voltage across the membrane but also by the ionic concentration gradients.

So far two terms have been used to indicate the ability of ions to cross membranes: permeability and conductance. Although they are related, they should not be confused. The permeability of a membrane to an ion is an intrinsic property of the membrane that is a measure of the ease with which the ion passes through the membrane (in units of cm/s). Permeability depends only on the types and numbers of ion channels present in the membrane. Conductance, on the other hand, measures the ability of the membrane (or channel) to carry electrical current (in units of 1/ohms). Since current is carried by ions, the conductance of a membrane will depend not only on the properties of the membrane but also on the concentration of ions in solution. A membrane can have a very high permeability to K⁺ ions, but if there is no K⁺ in solution there can be no K⁺ current flow and so the conductance of the membrane will be zero. In practice, permeability is used in the Goldman equation whereas conductance is used in electrical measurements and equivalent circuits.

A cell membrane has many resting K⁺ channels, all of which can be combined into a single equivalent circuit consisting of a conductor in series with a battery. All of the passive K⁺ channels in a nerve cell membrane can be lumped into a single equivalent electrical structure comprising a battery (E_{K}) in series with a conductor (g_{K}). The conductance is g_{K}=N_{K}×γ_{K} where N_{K} is the number of passive K⁺ channels and γ_{K} is the conductance of a single K⁺ channel. In this equivalent circuit the total conductance of all the K⁺ channels (g_{K}), i.e., the K⁺ conductance of the cell membrane in its resting state, is equal to the number N of resting K⁺ channels multiplied by the conductance of an individual K⁺ channel (γ_{K}):

g_{K}=N_{K}×γₖ

Since the battery in this equivalent circuit depends solely on the concentration gradient for K⁺ and is independent of the number of K⁺ channels, its value is the equilibrium potential for K⁺, E_{K}.

Using the Equivalent Circuit Model to Calculate Resting Membrane Potential

The equivalent circuit model of the resting membrane can be used to calculate the resting potential. To simplify the calculation we shall initially ignore Cl⁻ channels and begin with just two types of passive channels, K⁺ and Na⁺. In this example, the electrical equivalent circuit omits the Cl⁻ pathway and Na⁺—K⁺ pump for simplicity in calculating the resting membrane potential. Moreover, we ignore the electrogenic influence of the Na⁺—K⁺ pump because it is small. Because we will consider only steady-state conditions, where Vₘ is not changing, we can also ignore membrane capacitance. Because there are more passive channels for K⁺ than for Na⁺, the membrane conductance for current flow carried by K⁺ is much greater than that for Na⁺. In the equivalent circuit of this example, g_{K} is 20 times higher than g_{Na} (10×10⁻⁶ S compared to 0.5×10⁻⁶ S). Given these values and the values of E_{K} and E_{Na} the membrane potential, Vₘ is calculated as follows.

Since Vₘ is constant in the resting state, the net current must be zero, otherwise the separation of positive and negative charges across the membrane would change, causing Vₘ to change. Therefore I_{Na} is equal and opposite to I_{K}:

−I_{Na}=I_{K}

or

I_{Na}+I_{K}=0 (I)

We can easily calculate I_{Na} and I_{K} in two steps. First, we add up the separate potential differences across the Na⁺ and K⁺ branches of the circuit. Going from the inside to the outside across the Na⁺ branch, the total potential difference is the sum of the potential differences across E_{Na} and across g_{Na}:

Vₘ=E_{Na}+I_{Na}/g_{Na}

Similarly, for the K⁺ conductance branch:

Vₘ=E_{K}+I_{K}/g_{K}

Next, we rearrange and solve for I:

I_{Na}=g_{Na}×(Vₘ−E_{Na}) (II)

I_{K}=g_{K}×(Vₘ−E_{K}) (III)

As these equations illustrate, the ionic current though each conductance branch is equal to the conductance of the branch multiplied by the net electrical driving force. For example, the conductance for the K⁺ branch is proportional to the number of open K⁺ channels, and the driving force is equal to the difference between Vₘ and E_{K}. If Vₘ is more positive than E_{K} (−75 mV), the driving force is positive (outward); if Vₘ. is more negative than E_{K}, the driving force is negative (inward).

In Equation I above, we saw that I_{Na}+I_{K}=0. If we now substitute Equations II and III for I_{Na} and I_{K} in Equation I, multiply through, and rearrange, we obtain the following expression:

Vₘ×(g_{Na}+g_{K})=(E_{Na}×g_{Na})+(E_{K}×g_{K}).

(Because we have defined Vₘ as Vᵢₙ−Vₒᵤₜ, the following convention must be used for these equations. Outward current (in this case I_{K}) is positive and inward current is negative. Batteries with their positive poles toward the inside of the membrane (e.g. E_{Na}) are given positive values in the equations. The reverse is true for batteries that have their negative poles toward the inside, such as the K⁺ battery.)

Solving for Vₘ, we obtain an equation for the resting membrane potential that is expressed in terms of membrane conductances and batteries.

$${V}_{m} = \frac{( {E}_{Na} \times {g}_{Na} ) + ( {E}_{K} \times {g}_{K} )}{{g}_{Na} + {g}_{K}} \qquad ( IV )$$

From this equation, using the values in an equivalent circuit where g_{Na}=0.5×10⁻⁶ and g_{K}=10×10⁻⁶, and E_{Na}=+55 mV, and E_{K}=−75 mV, we calculate Vₘ=31 69 mV.

Equation IV states that Vₘ will approach the value of the ionic battery that is associated with the greater conductance. This principle can be illustrated by considering what happens during the action potential. At the peak of the action potential g_{K} is essentially unchanged from its resting value, but g_{Na} increases by as much as 500-fold. This increase in g_{Na} is caused by the opening of voltage-gated Na⁺ channels. In the equivalent circuit example described above, a 500-fold increase would change g_{Na} from 0.5×10⁻⁶ S to 250×10⁻⁶ S. If we substitute this new value of g_{Na} into Equation IV and solve for Vₘ, we obtain +50 mV, a value much closer to E_{K}. Vₘ is closer to E_{Na} than to E_{K} at the peak of the action potential because, since g_{Na} is now 25-fold greater than g_{K}, the Na⁺ battery becomes much more important than the K⁺ battery in determining Vₘ.

The real resting membrane has open channels not only for Na⁺ and K⁺, but also for Cl⁻. One can derive a more general equation for Vₘ following the steps outlined above, from an equivalent circuit that includes a conductance pathway for C⁻ with its associated Nernst battery:

$${V}_{m} = \frac{( {E}_{Na} \times {g}_{Na} ) + ( {E}_{K} \times {g}_{K} ) + ( {E}_{Cl} \times {g}_{Cl} )}{{g}_{Na} + {g}_{k} + {g}_{Cl}} \qquad ( V )$$

This equation is similar to the Goldman equation presented earlier. As in the Goldman equation, the contribution to Vₘ of each ionic battery is weighted in proportion to the conductance of the membrane for that particular ion. In the limit, if the conductance for one ion is much greater than that for the other ions, Vₘ will approach the value of that ion's Nernst potential.

The contribution of C⁻ ions to the resting potential can now be determined by comparing Vₘ calculated for the circuits for Na⁺ and K⁺ only, and for all three ions including Cl⁻. For most nerve cells the value of g_{Cl} ranges from one fourth to one-half of g_{K}. In addition, E_{Cl}, is typically quite close to E_{K}, but slightly less negative. In an example circuit, Cl⁻ ions are passively distributed across the membrane, so that E_{Cl} is equal to the value of Vₘ, which is determined by Na⁺ and K⁺. Note that if E_{Cl}=Vₘ (−69 mV in this example), no net current flows through the Cl⁻ channels. If we include g_{Cl} (2.5×10⁻⁶ S) in this example and E_{Cl} in the calculation of Vₘ the calculated value of Vₘ does not differ from that for the prior example. On the other hand, if Cl⁻ were not passively distributed but actively transported out of the cell, then E_{Cl}, would be more negative than −69 mV. Adding the Cl⁻ pathway to the calculation would then shift Vₘ to a slightly more negative value.

The equivalent circuit can be further simplified by lumping the conductance of all the resting channels that contribute to the resting potential into a single conductance g₁ and replacing the battery for each conductance channel with a single battery whose value, E₁, is that predicted by Equation V:

g₁=g_{Cl}+g_{Na}+g_{K}=13×10⁻⁶ S

$${E}_{1} = \frac{{g}_{K} {E}_{K} + {g}_{Cl} {E}_{Cl} + {g}_{Na} {E}_{Na}}{{g}_{Cl} + {g}_{Na} + {g}_{K}} = - 69 mV$$

This simplification will prove useful when the effects of gated channels are considered.

An Equivalent Circuit Model of the Membrane Includes Batteries, Conductors, a Capacitor, and a Current Generator

Like the population of resting K⁺ channels, all the resting Na⁺ channels can be represented by a single conductor in series with a single battery, as can the resting Cl⁻ channels. Since the K⁺, Na⁺, and C⁻ channels account for the bulk of the passive ionic current through the membrane in the cell at rest, the resting potential can be calculated by incorporating these three channels into a simple equivalent circuit of a neuron.

To construct this circuit we need only connect the elements representing each type of channel at their two ends with elements representing the extracellular fluid and cytoplasm. The extracellular fluid and cytoplasm are both excellent conductors because they have relatively large cross-sectional areas and many ions available to carry charge. Both can be approximated by a short circuit—a conductor with zero resistance.

The equivalent circuit of the neuron can be made more accurate by adding a current generator. Steady fluxes of Na⁺ and K⁺ ions through the passive membrane channels are exactly counterbalanced by active ion fluxes driven by the Na⁺—K⁺ pump, which extrudes three Na⁺ ions from the cell for every two K⁺ ions it pumps in. This electrogenic ATP-dependent pump, which keeps the ionic batteries charged, can be added to the equivalent circuit in the form of a current generator.

Under steady state conditions the passive Na⁺ and K⁺ currents are balanced by active Na⁺ and K⁺ fluxes (I′_{Na} and I′_{K}) driven by the Na⁺—K⁺pump. The lipid bilayer endows the membrane with electrical capacitance (Cₘ). Note I′_{Na} is 50% greater than I′_{K} (and therefore I_{Na} is 50% greater than I_{K}) since the Na⁺—K⁺ pump transports three Na⁺ ions out for every two K⁺ ions it transports into the cell.

Finally, we can complete the equivalent circuit of the neuron by incorporating its capacitance, the third important passive electrical property of the neuron. Capacitance is the property of an electric nonconductor (insulator) that permits the storage of charge when opposite surfaces of the nonconductor are maintained at a difference of potential. For the neuron, the nonconductor (or capacitor) is the cell membrane, which separates the cytoplasm and extracellular fluid, both of which are highly conductive environments. Strictly speaking, the membrane is a leaky capacitor because it is penetrated by ion channels. However, since the density of the ion channels is low, the insulating portion of the membrane—the lipid bilayer—occupies at least 100 times the area of all the ion channels combines.

The electrical potential difference across a capacitor, V, is expressed as:

V=Q/C

where Q is the excess of positive or negative charges on each side of the capacitor and C is the capacitance. Capacitance is measured in units of farads, F, where a charge separation of 1 coulomb across a 1 farad capacitor produces a 1 volt difference.

A typical value of membrane capacitance for a nerve cell is about 1 μF/cm² of membrane area. The excess of positive and negative charges separated by the membrane of a spherical cell body with a diameter of 50 μm and a resting potential of −60 mV is 29×10⁶ ions. Although this number may seem large, it represents only a tiny fraction (1/200,000) of the total number of positive or negative charges in solution within the cytoplasm. The bulk of the cytoplasm and the bulk of the extracellular fluid are electroneutral.

The use of the equivalent circuit model of the neuron to analyze neuronal properties quantitatively has been described above.

An Overall View

The lipid bilayer, which is virtually impermeant to ions, is an insulator separating two conducting solutions, the cytoplasm and the extracellular fluid. Ions can cross the lipid bilayer only by passing through ion channels in the cell membrane. When the cell is at rest, the passive ionic fluxes into and out of the cell are balanced, so that the charge separation across the membrane remains constant and the membrane potential remains at its resting value.

The value of the resting membrane potential in nerve cells is determined primarily by resting channels selective for K⁺, Cl⁻, and Na⁺. In general, the membrane potential will be closest to the equilibrium (Nernst) potential of the ion (or ions) with the greatest membrane permeability. The permeability for an ion species is proportional to the number of open channels that allow passage of that ion.

At rest, the membrane potential is close to the Nernst potential for K⁺, the ion to which the membrane is most permeable. The membrane is also somewhat permeable to Na⁺, however, and therefore an influx of Na⁺ drives the membrane potential slightly positive to the K⁺ Nernst potential. At this potential the electrical and chemical driving forces acting on K⁺ are no longer in balance, so K⁺ diffuses out of the cell. These two passive fluxes are each counterbalanced by active fluxes driven by the Na⁺—K⁺ pump.

Chloride is actively pumped out of some, but not all, cells. When it is not, it is passively distributed so as to be at equilibrium inside and outside the cell. Under most physiological conditions the bulk concentrations of Na⁺, K⁺, and C⁻ inside and outside the cell are constant. During signaling the changes in membrane potential (action potentials, synaptic potentials, and receptor potentials) are caused by substantial changes in the membrane's relative permeabilities to these three ions, not by changes in the bulk concentrations of ions, which are negligible. These changes in permeability, caused by the opening of gated ion channels, cause changes in the net charge separation across the membrane.

The embodiments of the invention, and the invention itself, are now described in such full, clear, concise and exact terms to enable a person of ordinary skill in the art to make and use the invention. To particularly point out and distinctly claim the subject matters regarded as invention, the following claims conclude this specification. To the extent variations from the preferred embodiments fall within the limits of the claims, they are considered to be part of the invention, and claimed.

What is claimed is:

1. A method of treating medical disorders comprising the step of modifying the ion concentration of brain fluid in the brain of a patient.

2. A method as in claim 1 of treating neurological disorders.

3. A method as in claim 2 further comprising the step of measuring the ion concentration of the brain fluid.

4. A method as in claim 2 further comprising modifying the ion concentration of brain fluid by delivering fluid to the brain, the ion concentration of the delivered fluid being such as to cause the ion concentration in the brain to be modified.

5. A method as in claim 4 further including replacing brain fluid.

6. A method as in claim 5 further including replacing brain fluid with brain fluid previously extracted from the brain.

7. A method as in claim 4 in which the step of delivering fluid to the brain includes delivering modulated ion-content fluid into the patient's brain into at least one localized region of the patient's brain.

8. A method as in claim 4 in which the step of delivering fluid to the brain includes pumping modulated ion-content fluid into the patient's brain according to a predetermined schedule of flow rate.

9. A method as in claim 4 in which the step of delivering fluid to the brain includes delivering modulated ion-content fluid into the patient's brain into a general region of the patient's brain.

10. A method as in claim 4 further comprising measuring ion concentration in said brain fluid after beginning said delivering of fluid to the brain, and adjusting the ion concentration of the delivered fluid based on the measured ion concentration.

11. The method of claim 4, further comprising the steps of calculating ion concentration in said brain fluid, using a membrane potential equation, and adjusting the delivery of modulated ion-content fluid, based on the calculated ion concentration.

12. The method of claim 11, wherein the membrane potential equation is the Goldman equation or a derivative or modification of the Goldman equation.

13. The method of claim 4, further comprising the steps of measuring the electrical conductivity of said brain fluid after delivering modulated ion-content fluid to the patient's brain, and adjusting the delivery of said modulated ion-content fluid, based on the measured electrical conductivity of said brain fluid.

14. The method of claim 4, wherein the delivery is of modulated ion-content fluid and said delivery is calculated using a membrane potential equation.

15. The method of claim 14, wherein the membrane potential equation is the Goldman equation or a derivative or modification of the Goldman equation.

16. The method of claim 4 wherein the delivery is of modulated ion-content fluid and the delivered fluid produces a voltage differential between intra-cellular fluid and extra-cellular fluid, which may be modified to such a level that epileptic seizures are controlled.

17. The method of claim 4 wherein the fluid is delivered to the patient's brain using closed-loop feedback.

18. The method of claim 4 further wherein the delivery is of modulated ion-content fluid and comprised of the step of adjusting the delivery of modulated ion-content fluid based upon the measured electrical activity of predetermined most likely epileptic brain cells.

19. The method of claim 4 wherein said delivery is of modulated ion-content fluid and said fluid is delivered to the brain using a dispersed delivery system.

20. The method of claim 4 wherein said delivery is of modulated ion-content fluid and said fluid is delivered to the brain ventricle.

21. The method of claim 4 wherein said delivery is of modulated ion-content fluid and said fluid is delivered to the brain using a computer-controlled pump.

22. The method of claim 4 wherein said delivery is of modulated ion-content fluid and said fluid is delivered to the brain at a predetermined location by direct injection into a localized region.

23. A system for controlling epileptic seizures comprising:
a) a brain fluid pumping mechanism, having an input, coupled to a patient's brain for extracting brain fluid, and having an output;
b) a fluid ion adjustment mechanism coupled to said output of said brain fluid pumping mechanism, said fluid ion adjustment mechanism having an output from which modulated ion-content fluid is produced; and
c) a catheter, having an input coupled to the output of said ion adjustment mechanism and having an output inserted into a predetermined region of a patient's brain,
whereby brain fluid is extracted from a patient's brain, ion-concentration of said fluid is adjusted and said brain fluid is re-injected into said brain.

24. A system as in claim 23 in which the system includes computer control that reads and executes stored program instructions that cause the pumping mechanism to pump the extracted fluid according to the program and its parameters.

25. A system as in claim 23 further comprising a probe in the brain providing an output related to measurement of an ion-concentration related brain parameter, the system including computer control which reads the output of the probe to responsively control at least one of fluid extraction, fluid delivery, and ion concentration.

26. A system as in claim 25 in which the computer control calculates ion concentration in brain fluid using the Goldman equation, and adjusts the delivery of modulated ion-content fluid based on the calculated ion concentration.

27. A system as in claim 25 in which the computer control calculates ion concentration in brain fluid using a membrane potential equation.

28. A system as in claim 27 in which the membrane potential equation is the Goldman equation or a derivative or a modification of the Goldman equation.

29. A system for controlling epileptic seizures comprising:
a) a fluid pumping mechanism, having an input, coupled to a fluid source selected from the group consisting of a patient's brain and a source other a patient's brain, and having an output;
b) a fluid ion adjustment mechanism coupled to said output of said brain fluid pumping mechanism, said fluid ion adjustment mechanism having an output from which modulated ion-content fluid is produced; and
c) a catheter, having an input coupled to the output of said ion adjustment mechanism and having an output inserted into a predetermined region of a patient's brain, whereby modulated ion-content fluid can be injected into the brain.

30. The apparatus of claim 29 wherein output of the catheter includes a fluid passageway to provide modulated ion-content fluid into the patient's brain into at least one localized region of the patient's brain.

31. The apparatus of claim 29 wherein the fluid pumping mechanism includes means for pumping the modulated ion-content fluid into the patient's brain according to a predetermined flow rate.

32. The apparatus of claim 29 wherein output of the catheter includes a fluid passageway to provide modulated ion-content fluid into the patient's brain into a general region of the patient's brain.

33. The apparatus of claim 29 further comprising means for measuring ion concentration in the brain fluid after the modulated ion-content fluid is injected into the patient's brain.

34. The apparatus of claim 33 wherein the fluid pumping mechanism or fluid adjustment mechanism includes means for adjusting the delivery of the modulated ion-content fluid based on the measured ion concentration.

35. The apparatus of claim 29 further comprising:
means for calculating ion concentration in the brain fluid using a membrane potential equation;
the fluid pumping mechanism or fluid ion adjustment mechanism including means for adjusting the delivery of the modulated ion-content fluid based upon the calculated ion concentration.

36. The apparatus of claim 29 further comprising means for measuring the electrical conductivity of the brain fluid after the modulated ion-content fluid is injected into the patient's brain; the fluid pumping mechanism or fluid ion adjustment mechanism including means for adjusting the delivery of the modulated ion-content fluid, based upon the measured electrical conductivity of the brain fluid.

37. The apparatus of claim 36 wherein the means for measuring the electrical conductivity of the brain fluid after the modulated ion-content fluid is injected into the patient's brain comprises an electrical probe configured and adapted for insertion into brain fluid to measure conductivity or resistance of brain fluid.

38. The apparatus of claim 35 wherein the membrane potential equation is the Goldman equation or a derivative or modification of the Goldman equation. calculating the ion concentration of the brain fluid using the Goldman equation.

39. The apparatus of claim 36 wherein the ion fluid adjustment mechanism is adapted such that the modulated ion-content fluid injected into the brain produces a voltage differential between intra-cellular fluid and extra-cellular fluid that is modified to such a level that epileptic seizures are controlled.

40. The apparatus of claim 39 further comprising closed-loop feedback means for delivery of the modulated ion-content fluid to the patient's brain.

41. The apparatus of claim 29 further comprising means for measuring electrical activity of predetermined most likely epileptic brain cells, the fluid pumping mechanism or fluid ion adjustment mechanism including means for adjusting the delivery of the modulated ion-content fluid based upon measured electrical activity of predetermined most likely epileptic brain cells.

42. The apparatus of claim 29 wherein the catheter comprises a dispersed delivery system for injecting modulated ion-content fluid to the patient's brain.

43. The apparatus of claim 29 wherein the catheter is configured and adapted to provide the modulated ion-content fluid to a brain ventricle.

44. The apparatus of claim 29 wherein the fluid pumping mechanism comprises computer-controlled pump.

45. The apparatus of claim 29 wherein the catheter is configured and adapted to provide the modulated ion-content fluid to the brain at a predetermined location by direct injection into a localized region.

46. The apparatus of claim 29 which the fluid ion adjustment mechanism includes an ion exchange mechanism to adjust fluid ion concentration.

47. The apparatus of claim 46 in which the ion exchange mechanism comprises means for filtering fluid to adjust fluid ion concentration.

48. The apparatus of claim 46 in which the ion exchange mechanism comprises chemical means for treating fluid to adjust ion concentration.

49. Apparatus for treating epilepsy and other neurological disorders of the brain comprising:
means for modifying ion concentrations of a fluid to render modulated ion-content fluid using a predetermined process;
means for substantially continuously pumping the modulated ion-content fluid into a localized region of the patient's brain;
means for monitoring the ion concentration of brain fluid proximate to the region where the ion-content fluid is pumped to the patient's brain.

50. The apparatus of claim 49 further comprising means for diagnosing an epileptic condition in a patient.
